(19) United States
(12) Patent Application Publication
(10) Pub. No.: US 2025/0049331 A1
(43) Pub. Date: Feb. 13, 2025

(54) Estimating Blood Pressure Using PPG Traces

(71) Applicant: Artilux, Inc., Menlo Park, CA (US)

(72) Inventor: Konstantin P. Petrov, HSINCHU COUNTY (TW)

(21) Appl. No.: 18/800,752

(22) Filed: Aug. 12, 2024

Related U.S. Application Data
(60) Provisional application No. 63/519,030, filed on Aug. 11, 2023.

(51) Int. Cl. A61B 5/021; A61B 5/00

(52) U.S. Cl. CPC A61B 5/02108; A61B 5/7225; A61B 5/7235; A61B 5/7278; A61B 5/742; A61B 2560/0223; A61B 2560/0462

(57) ABSTRACT

Methods and systems for estimating a blood pressure of a user of a computing device are disclosed herein. The method can include obtaining at least one calibration trace, the at least one calibration trace being constructed to yield a fit between one or more selected reference traces and a corresponding parameter reference value for each of the one or more selected reference traces and obtaining a photoplethysmography (PPG) trace associated with a heartbeat cycle. The method can also include determining a convolution of the PPG trace with the at least one calibration trace, determining a blood pressure estimate for the user based on the convolution of the PPG trace with the at least one calibration trace, and providing data representing the blood pressure estimate.

### CROSS-REFERENCE TO RELATED APPLICATIONS

This application claims benefit of U.S. Provisional Patent Application Ser. No. 63/519,030, entitled “Estimating Blood Pressure Using PPG Traces” filed on Aug. 11, 2023, which is incorporated by reference herein in its entirety.

### FIELD

The present disclosure relates generally to estimating blood pressure for a user of a computing device. In particular, the present disclosure relates to estimating blood pressure of a user of a wearable computing device, such as a smart watch and/or activity tracker, by using photoplethysmography (“PPG”) traces from the user.

### BACKGROUND

Today's prevailing method of blood pressure estimation using PPGs involves extraction of “cardiologically significant” (“CLS”) parameters from human heartbeat cycles represented in the PPG traces. Correlation models use these parameters, obtained from large amounts of data, to attempt to accurately predict systolic blood pressure (“SBP”) and diastolic blood pressure (“DBP”). However, difficulties can arise in fitting thousands of instances of blood pressure values using these parameters, as only a relative few parameters (e.g., a dozen) are postulated to be impactful on the blood pressure determination. Additionally, the data (e.g., blood pressure values) used by these models represent a broad range of patients, including those suffering from cardiovascular ailments or trauma or other disorders, resulting in a broad spectrum of PPG signals. It therefore can be challenging to fit these CLS parameters to the blood pressure values, with the resulting fit having a high mean absolute error.

### SUMMARY

Aspects and advantages of embodiments of the present disclosure will be set forth in part in the following description, or may be learned from the description, or may be learned through practice of the embodiments.

One example aspect of the present disclosure is directed to a method for estimating a blood pressure of a user of a computing device. The method includes obtaining, by one or more processors, at least one calibration trace, the at least one calibration trace being constructed to yield a fit between one or more selected reference traces and a corresponding parameter reference value for each of the one or more selected reference traces and obtaining, by the one or more processors, a photoplethysmography (PPG) trace associated with a heartbeat cycle. The method also includes determining, by the one or more processors, a convolution of the PPG trace with the at least one calibration trace and determining, by the one or more processors, a blood pressure estimate for the user based on the convolution of the PPG trace with the at least one calibration trace. The method further includes providing, by the one or more processors, data representing the blood pressure estimate.

In some implementations, the computing device is a wearable computing device comprising a sensor, and the sensor obtains the PPG trace.

In some implementations, the PPG trace is equal in size to the at least one calibration trace.

In some implementations, the PPG trace includes a plurality of trace points, and wherein the plurality of trace points are sampled at arbitrarily chosen locations in the heartbeat cycle.

In some implementations, the at least one calibration trace includes two calibration traces. The two calibration traces can include (i) a first calibration trace associated with systolic blood pressure values, and (ii) a second calibration trace associated with diastolic blood pressure values.

In some implementations, the blood pressure estimate comprises an output from the convolution of the PPG trace and at least one of the first calibration trace and the second calibration trace.

In some implementations, the PPG trace includes a plurality of trace points. The method can further include determining, by the one or more processors, a weighted value for each trace point of the plurality of trace points based on the convolution, determining, by the one or more processors, a region of interest in the PPG trace based on the determined weighted value for each trace point of the plurality of trace points, and determining, by the one or more processors, a group of sampling points having a higher sampling rate in the region of interest for a future PPG trace.

In some implementations, each calibration trace of the at least one calibration trace comprises a linear array of calibration coefficients representing weights of points associated with the one or more selected reference traces.

In some implementations, the method can also include presenting the data to the user via a display of the computing device.

Another example embodiment of the present disclosure is directed to a computing device for estimating a blood pressure of a user. The computing device can include one or more processors and a memory storing instructions for execution by the one or more processors to cause the one or more processors to perform operations. The operations can include obtaining at least one calibration trace, the at least one calibration trace being constructed to yield a fit between one or more selected reference traces and a corresponding parameter reference value for each of the one or more selected reference traces and obtaining a photoplethysmography (PPG) trace associated with a heartbeat cycle. The operations can also include determining a convolution of the PPG trace with the at least one calibration trace and determining a blood pressure estimate for the user based on the convolution of the PPG trace with the at least one calibration trace. The operations can further include providing data representing the blood pressure estimate.

In some implementations, the computing device is a wearable computing device and further comprises a sensor, wherein the sensor obtains the PPG trace.

In some implementations, the PPG trace includes a plurality of trace points, and wherein the plurality of trace points are sampled at arbitrarily chosen locations in the heartbeat cycle.

In some implementations, the at least one calibration trace includes two calibration traces. The two calibration traces can include (i) a first calibration trace associated with systolic blood pressure values, and (ii) a second calibration trace associated with diastolic blood pressure values.

In some implementations, the blood pressure estimate comprises an output from the convolution of the PPG trace and at least one of the first calibration trace and the second calibration trace.

In some implementations, the PPG trace includes a plurality of trace points. The operations can further include determining a weighted value for each trace point of the plurality of trace points based on the convolution, determining a region of interest in the PPG trace based on the determined weighted value for each trace point of the plurality of trace points, and determining a group of sampling points having a higher sampling rate in the region of interest for a future PPG trace.

A further example aspect of the present disclosure is directed to a non-transitory, computer-readable medium storing instructions for execution by one or more processors to cause the one or more processors to perform operations. The operations can include obtaining at least one calibration trace, the at least one calibration trace being constructed to yield a fit between one or more selected reference traces and a corresponding parameter reference value for each of the one or more selected reference traces and obtaining a photoplethysmography (PPG) trace associated with a heartbeat cycle. The operations can also include determining a convolution of the PPG trace with the at least one calibration trace and determining a blood pressure estimate for a user based on the convolution of the PPG trace with the at least one calibration trace. The operations can further include providing data representing the blood pressure estimate.

In some implementations, the data representing the blood pressure estimate is provided by a wearable computing device that further comprises a sensor, wherein the sensor obtains the PPG trace.

In some implementations, the PPG trace is equal in size to the at least one calibration trace.

In some implementations, the at least one calibration trace includes two calibration traces, the two calibration traces including a first calibration trace associated with systolic blood pressure values and a second calibration trace associated with diastolic blood pressure values.

In some implementations, the PPG trace includes a plurality of trace points. The operations can further include determining a weighted value for each trace point of the plurality of trace points based on the convolution, determining a region of interest in the PPG trace based on the determined weighted value for each trace point of the plurality of trace points, and determining a group of sampling points having a higher sampling rate in the region of interest for a future PPG.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

The foregoing aspects and many of the advantages of this application will become more readily appreciated as the same becomes better understood by reference to the following detailed description, when taken in conjunction with the accompanying drawings:

FIG. 1 illustrates a block diagram of a wearable computing device according to example embodiments of the present disclosure.

FIG. 2 illustrates a flow chart of a method for estimating a blood pressure of a user according to example embodiments of the present disclosure.

FIG. 3 illustrates a sampled PPG trace according to example embodiments of the present disclosure.

FIG. 4 illustrates a result of a convolution between a PPG trace and a calibration trace according to example embodiments of the present disclosure.

FIG. 5 illustrates example computing system components and devices according to example aspects of the present disclosure.

FIG. 6 illustrates a first example sensor according to example aspects of the present disclosure.

FIG. 7 illustrates a second example sensor according to example aspects of the present disclosure.

### DETAILED DESCRIPTION

The present disclosure is directed to estimating blood pressure of a user. Photoplethysmography (“PPG”) traces can be gathered and cardiologically significant (“CLS”) parameters can be extracted through analysis of these traces. It can be observed that CLS parameters are linear combinations of different subsets of the same discrete data set of these traces, and these subsets inevitably overlap because some CLS parameters require integral and derivative computation on this discrete data. Because of this, these CLS parameters in their discrete approximation cannot in principle be mathematically independent from one another; in other words, an explicit linear combination can be written that is identically zero.

The present disclosure dispenses with direct utilization of only CLS parameters in favor of treating all PPG trace points in a uniform manner as potentially equally informative by computing the blood pressure estimate as a fixed linear combination of all available trace points within a single heartbeat cycle, each weighted by an individual calibration coefficient. This can be equivalent to a discrete convolution of the heartbeat trace with another, hypothetical, pre-computed, fixed, synchronous calibration trace. Systolic blood pressure (SBP) and diastolic blood pressure (DBP) are computed independently by convolving the same measured PPG trace with two different calibration traces.

The calibration trace includes a calibration vector and can be constructed to yield a particular fit (e.g., a best fit) between a particular quantity (e.g., a number N) of chosen measured reference traces and corresponding independently measured parameter reference values. In some embodiments, the calibration vector can include a linear array of calibration coefficients representing respective weights of points associated with reference traces. In the example of calculating blood pressure, the calibration vector can then be convolved with the PPG trace to estimate the blood pressure of the user. For example, the PPG trace can include M trace points sampled at M arbitrary locations of each PPG trace, where M is less than N reference traces in a database. With a sufficiently large database, all available heartbeat trace (e.g., PPG trace) points can be used for calculation of blood pressure, which maximizes the number of degrees of freedom available for fitting the measured blood pressure values within a statistical model. Additionally, the use of the calibration trace automatically determines which particular sections of the PPG trace are more statistically significant than others in terms of their impact on blood pressures. This allows for operation at a reduced or variable sampling rate that focuses on mapping sampling to these particular sections while the fitting can maintain a low mean absolute error (MAE).

The pre-computation of calibration traces requires only an inversion of an M*M real-number matrix and does not require any proprietary software, high-performance cloud-based computing resources, neural networks, or other artificial intelligence. The required bandwidth and processing power can therefore be realized within a battery-powered wearable device such as a smart watch or activity monitoring bracelet.

With reference to the figures, example embodiments of the present disclosure will be discussed in further detail. It should be understood that embodiments, features, hardware, software, and/or other elements described with respect to one figure can be utilized within the systems and processes of another figure.

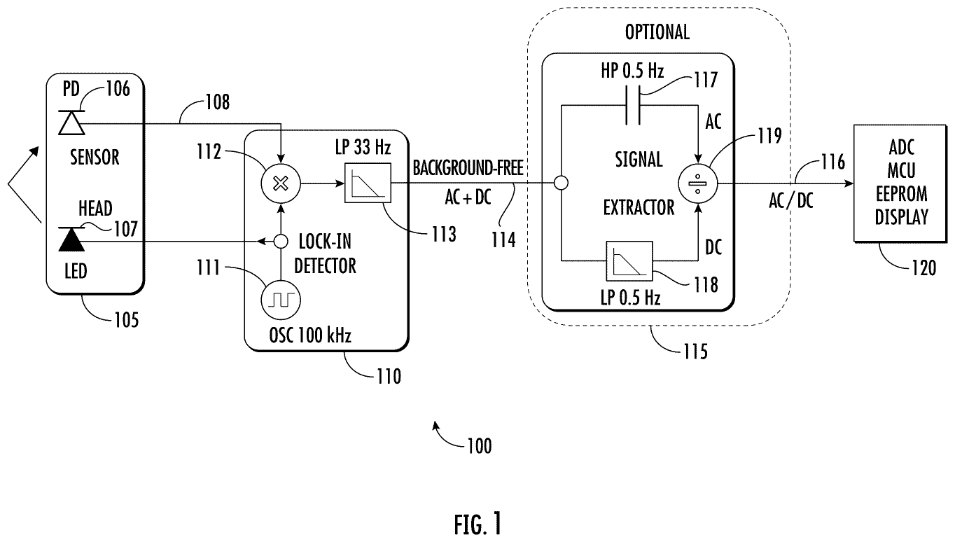

FIG. 1 is a block diagram illustrating a wearable computing device 100 according to example embodiments of the present disclosure. The wearable computing device 100 can include a sensor 105, a lock-in detector 110, a signal extractor 115, and device components 120. Although the present disclosure describes different components implemented on a single wearable computing device 100, in some implementations different components described below may be implemented on multiple devices (e.g., the signal extractor 115 and/or the device components 120 may be implemented on a separate device).

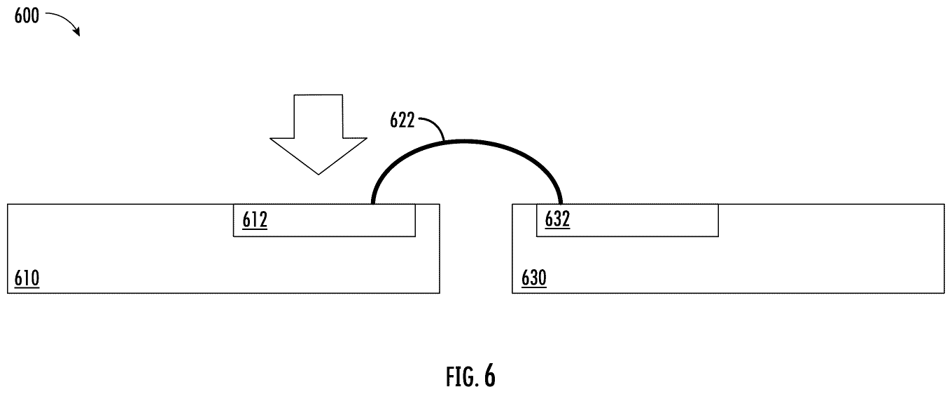

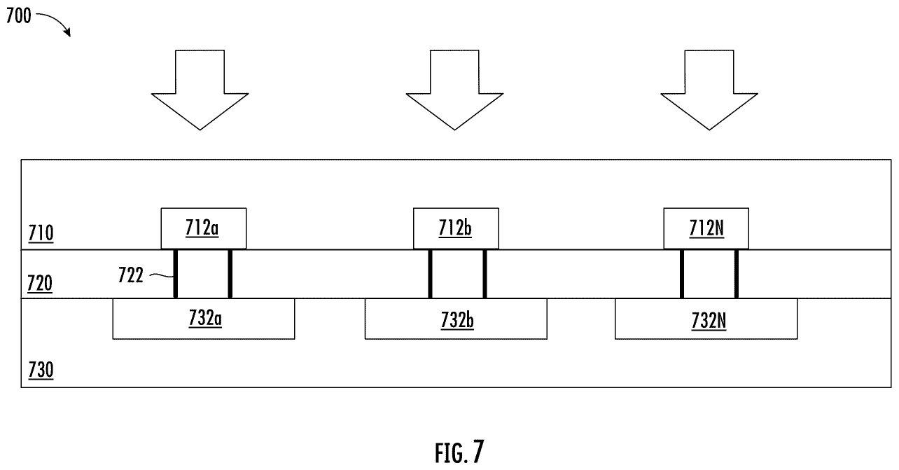

The sensor 105 can be a sensor such as a pulse transit time sensor, an optical sensor for gathering biological data about the user, or another appropriate sensor that can gather sensor data 108 related to cardiovascular activity of the user. In some embodiments, the sensor 105 can include one or more photodetectors (PDs) 106 (e.g., photodetectors 600 and 700 as described in reference to FIGS. 6 and 7) and/or LEDs 107 configured to operate as light-based optical sensor components. In some embodiments, the sensor 105 can gather cardiovascular activity data about the user and generate a sensor data 108 including a PPG trace based on the gathered data. Sensor data 108 obtained from sensor 105 can be provided from sensor 105 to lock-in detector 110.

The lock-in detector 110 can be used to extract a signal, such as a PPG trace, from data obtained by the sensor 105. In some embodiments, the lock-in detector 110 can be a component of the sensor 105 and the sensor 105, using the lock-in detector 110, can extract a signal from sensor data 108 detected by the sensor 105. The lock-in detector 110 can extract signal amplitudes and phases in noisy environments by using homodyne detection and low-pass filtering to measure signal amplitude and phase relative to a periodic reference clock. For example, lock-in detector can include an oscillator 111, a multiplier 112 and a low-pass (LP) filter 113. Oscillator 111 can be configured to generate an oscillation signal at a particular frequency (e.g., 100 kHz) or range of frequencies. Oscillator 111 can be coupled to and configured to provide the generated oscillation signal as a periodic reference clock to LED 107 of sensor 105 and to multiplier 112 of lock-in detector 110. Multiplier 112 can receive sensor data 108 from sensor 105 and the oscillation signal from oscillator 111 and combine the two via two-quadrant multiplication or other suitable combinatory technique. An output of multiplier 112 can be provided to LP filter 113 (e.g., a filter configured to pass signal frequencies less than about 33 Hz) to eliminate the background noise, which can output the determined signal 114 (e.g., the PPG trace) for use by other components of the wearable computing device 100. Determined signal 114 can be provided from lock-in detector 110 to a signal extractor 115. The determined signal 114 can include AC portion and DC portion of the PPG trace without the background noise.

The signal extractor 115 can receive the determined signal 114 output from lock-in detector 110 and further refine the received determined signal 114 into a trace 116, such as a PPG trace, for later analysis and processing. Signal extractor 115 can include, for example, an AC branch and a DC branch. The AC branch of signal extractor 115 can include one or more high-pass (HP) circuit elements 117, such as but not limited to a high-pass filter (e.g., a capacitor configured to pass signal frequencies greater than about 0.5 Hz). The DC branch of signal extractor 115 can include one or more low-pass (LP) circuit elements 118, such as but not limited to a low-pass filter (e.g., a low-pass filter configured to pass signal frequencies less than about 0.5 Hz). An AC output of the HP circuit elements 117 and a DC output of the LP circuit elements 118 can be provided to divider 119. Trace 116 generated by the divider 119 can be the AC output divided by the DC output and then be provided to one or more additional device components 120. Device components 120 can include an analog-to-digital converter, a main control unit (e.g., one or more electronic processors), a non-transitory, computer-readable medium (e.g., an EEPROM), and a display.

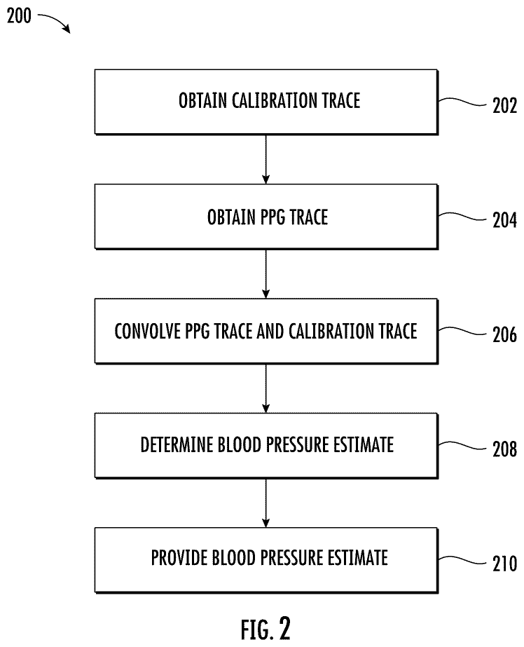

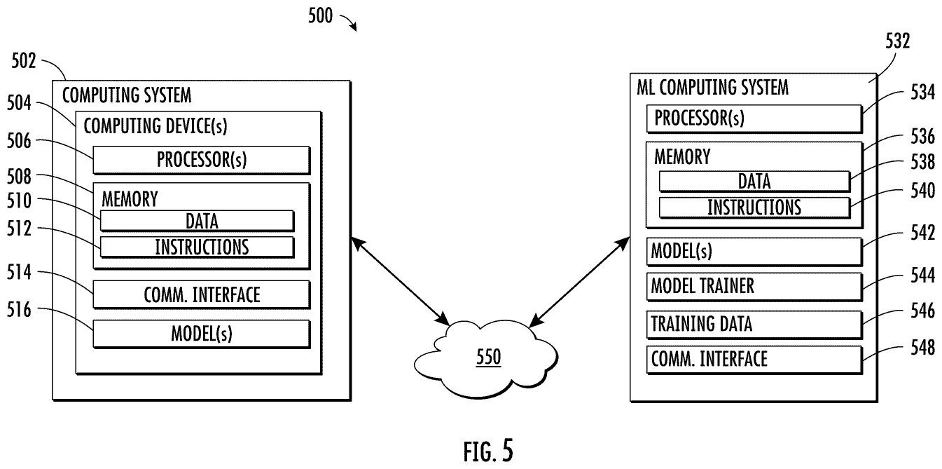

FIG. 2 illustrates a flow chart of a method 200 for estimating a blood pressure of a user according to example embodiments of the present disclosure. One or more portions of the method 200 can be implemented by one or more devices such as, for example, any of those described herein (e.g., computing devices, wearable computing devices, etc.). One or more portions of the method 200 described herein can be implemented as an algorithm on the hardware components of the devices described herein (e.g., as in FIGS. 1 and 5) to, for example, estimate blood pressure of a user. Although FIG. 2 depicts steps performed in a particular order for purposes of illustration and discussion, method 200 of FIG. 2 is not limited to the particularly illustrated order or arrangement. The various steps of the method disclosed herein can be omitted, rearranged, combined, and/or adapted in various ways without deviating from the scope of the present disclosure.

At 202, a computing system obtains at least one calibration trace, the at least one calibration trace being constructed to yield a fit between one or more selected reference traces (e.g., a database of PPG traces) and a corresponding parameter reference value (e.g., the blood pressure of a user) for each of the one or more selected reference traces.

The at least one calibration trace can be determined at 202 before any PPG traces are analyzed. For example, each reference trace can include M points sampled at M arbitrary locations of each reference trace. The at least one calibration trace is constructed to yield a best fit between N chosen measured reference traces (selected from, for example, a database of PPG traces), represented by a rectangular M*N matrix of trace reference values yₘⁿ, and the corresponding independently measured parameter reference values Y, (an N*1 vector).

In some embodiments, the at least one calibration trace can include a first calibration trace associated with SBP and a second calibration trace associated with DBP. Based on the gathered PPG trace, one of the two calibration traces can be selected to convolve with the PPG trace, as described below.

At 204, the computing system obtains a PPG trace associated with a heartbeat cycle. In some embodiments, the PPG trace includes a plurality of trace points and can be received from a sensor (e.g., sensor 105 shown in FIG. 1) of the computing system.

At 206, the computing system determines a convolution of the PPG trace with the at least one calibration trace.

A trace (e.g., PPG trace), or time-dependent function ƒ(t), can be sampled at M arbitrarily chosen locations (e.g., arbitrarily chosen trace points), and an M*1 matrix of data points (referred to herein as a “column” or a “vector”) is formed. From these data points, scalar parameters ε_{ƒ} associated with the trace, such as blood pressure, can have values estimated by convolving the vector ƒ with an equally-sized calibration vector c. The scalar parameters ε_{ƒ} can be obtained using Equation 1.

$${ε}_{f} = \hat{f} * c = {\sum }_{m = 1}^{M} {\hat{f}}^{m} {c}_{m} = {\sum }_{m = 1}^{M} {\hat{c}}^{m} {f}_{m} = \hat{c} * f , 1 * 1 = ( 1 * M ) * ( M * 1 ) \qquad Equation 1$$

Applying Equation 1 to each reference trace y and its corresponding parameter reference values Y and summing all squared errors leads to Equation 2. In addition, the sum of squared errors should be minimum.

$${\vert Y - \hat{y} * c \vert}^{2} = {\sum }_{n = 1}^{N} {\{ {Y}_{n} - {\sum }_{m = 1}^{M} {\hat{y}}_{n}^{m} {c}_{m} \}}^{2} = minimum \qquad Equation 2$$

Taking a partial derivative of both sides of Equation 2 with respect to cₖ for every k=1 . . . M, and taking note of the Kronecker delta δᵢₖ, M equations (represented by Equation 3) can be obtained.

$${\sum }_{n = 1}^{N} [ \{ {Y}_{n} - {\sum }_{m = 1}^{M} {\hat{y}}_{n}^{m} {c}_{m} \} {\sum }_{i = 1}^{M} \frac{\partial {c}_{i}}{\partial {c}_{k}} {\hat{y}}_{n}^{i} ] = {\sum }_{n = 1}^{N} [ \{ {Y}_{n} - {\sum }_{m = 1}^{M} {\hat{y}}_{n}^{m} {c}_{m} \} {\sum }_{m = 1}^{M} {δ}_{ik} {\hat{y}}_{n}^{i} ] = {\sum }_{n = 1}^{N} [ \{ {Y}_{n} - {\sum }_{m = 1}^{M} {\hat{y}}_{n}^{m} {c}_{m} \} {y}_{k}^{n} ] = 0 \qquad Equation 3$$

The independent summation index i=1 . . . M appears because of the products between sums that emerge during differentiation. Equation 3 can be rearranged, the order of summation can be swapped, and other identities can be applied to reach a set of equations represented by Equation 4.

$$\begin{matrix}{( y * Y )}_{k} = {\sum }_{n = 1}^{N} {y}_{k}^{n} {Y}_{n} = {\sum }_{m = 1}^{M} [ {c}_{m} {\sum }_{n = 1}^{N} {y}_{k}^{n} {\hat{y}}_{n}^{m} ] = {\sum }_{m = 1}^{M} {( y * \hat{y} )}_{k}^{m} {c}_{m} = {( y * \hat{y} * c )}_{k} \\ c = {( y * \hat{y} )}^{- 1} * ( y * Y ) , M * 1 = ( M * N * N * M ) * ( M * N * N * 1 )\end{matrix} \qquad Equation 4$$

$$\begin{matrix}{ε}_{f} = \hat{f} * c = \hat{f} * {( y * \hat{y} )}^{- 1} * ( y * Y ) , \\ 1 * 1 = ( 1 * M ) * ( M * N * N * M ) * ( M * N * N * 1 )\end{matrix}$$

From the structure of the y*ŷ matrix, it follows that its determinant is zero whenever N<M, and such a matrix cannot be inverted. Therefore, it is required that N (the number of reference traces) must be greater than M (the number of sample points). With a sufficiently large number of reference traces N, it is possible to sample each trace at more locations M. Each sampled trace point in the convolution ε_{ƒ}=\hat{ƒ}*c is a measured quantity that is mathematically independent of any other trace points, and there is no functional relationship tying any of them together.

With a database configured to store N reference traces, all available heartbeat (e.g., PPG) trace points ƒₘ can be used for calculation of the blood pressure estimate, thus ensuring 100% data utilization. This maximizes the number of degrees of freedom for fitting the measured blood pressure values within a statistical model.

This discrete linear convolution ε_{ƒ}=\hat{ƒ}*c enables each data point in the PPG trace to be functionally independent of all other data points and all data points can be treated equally.

At 208, the computing system determines a blood pressure estimate for the user based on the convolution of the PPG trace with the at least one calibration trace. The result of the convolution is a single parameter value for the PPG trace that is equivalent to an estimate of the blood pressure of the user.

In some embodiments, the computing system can determine a calibration weight for each trace point of the plurality of trace points based on the convolution. The calibration weights can be determined based on the convolution and indicate which portions of the PPG have more relevance (e.g., a higher calibration weight) to the blood pressure measurement.

In some embodiments, the computing system can determine a region of interest in the PPG trace based on the determined calibration weight for each trace point of the plurality of trace points. The region of interest can include, for example, a number of points within a set range within the PPG trace that have relatively high calibration weights. This indicates that the particular region of interest of the PPG trace is highly relevant to the final blood pressure estimate.

In some embodiments, the computing system can group sampling points for a future PPG trace in the region of interest. In other words, because the region of interest is highly relevant to the final blood pressure estimate, additional points in the region of interest being sampled can lead to a better fitted final blood pressure estimate.

At 210, the computing system can provide data representing the blood pressure estimate to a variety of sources, such as providing the data to the user via a display of the computing device.

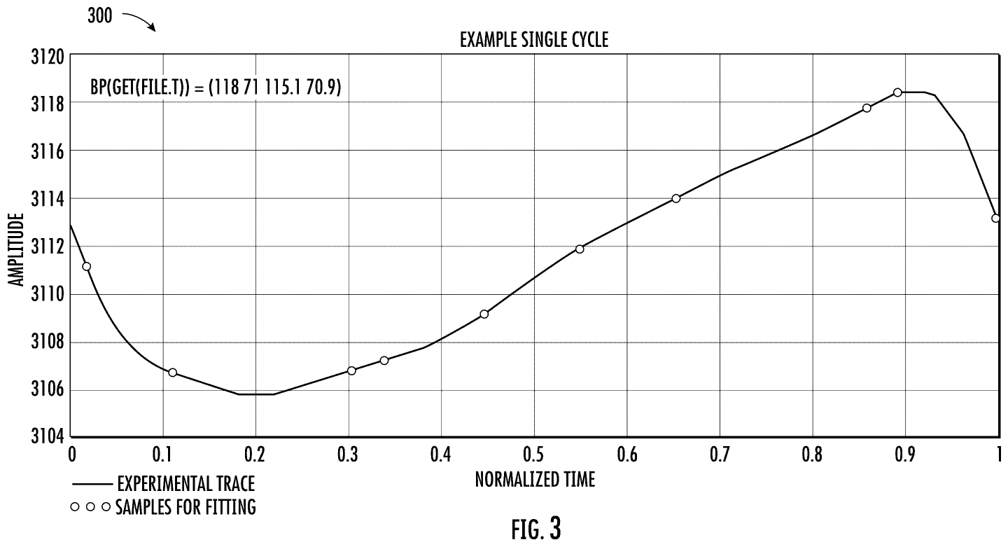

FIG. 3 illustrates a sampled PPG trace 300 according to example embodiments of the present disclosure. PPG trace 300 is made up of a plurality of trace points measured in a single heartbeat cycle. In this instance, the number of reference traces N is equal to 18, and the number of sampled points M is 12. Sampling locations can be arbitrary, but the same sampling location is applied to all measured traces. The PPG trace 300 depicted in FIG. 3 can correspond to a PPG trace obtained by the wearable computing device 100 of FIG. 1.

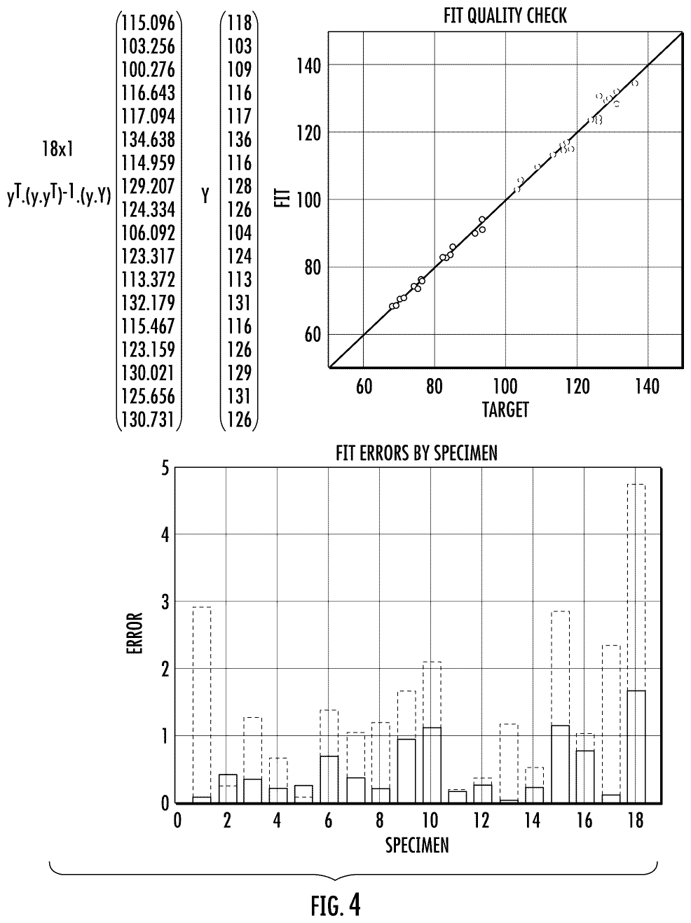

A vector Y is the vector of measured systolic blood pressures in unit mmHg, and y is a rectangular matrix of sampled PPG trace values. Values yₘⁿ in the rectangular matrix can include heart rate, offset normalized amplitude, and ten sampled values of the normalized heartbeat cycle in the trace 300. All elements of y are dimensionless real numbers from 0-1 with the exception of heart rate (can locate in the first row of y and measured in beats per minute, bpm). y is convolved with Y to obtain the calibration vector c for calculating of systolic blood pressure. Calibration vector c has a first element with units of mmHg/bmp, while the remaining elements are in mmHg. Every column of y is convolved with c as an estimated result, which is demonstrated in FIG. 4. The first quantity represents the systolic blood pressures estimated by convolution, and the second quantity is the measured systolic blood pressures. The values of the first quantity and the second quantity are very close. The mean absolute error (MAE) between the two can be less than 1 mmHg, and the worst MAE is around 4.7 mmHg. This result shows that the estimation method is sufficiently accurate. The results of the convolution illustrate the comparative importance of each particular trace data point in the determination of blood pressure, and the resulting values indicating the comparative importance of the particular trace data points are equivalent to the calibration weights for the trace data points. The resulting mapping can also indicate which particular regions of the heartbeat cycle are more informative for determining the blood pressure and therefore warrant more detailed sampling. In future PPG traces, sampling can be preferentially performed in determined regions of interest in the PPG traces instead of using a uniform sampling grid, which allows for fit quality to be improved without resorting to additional degrees of freedom. A computing system can determine a group of sampling points having a higher sampling rate in the region of interest.

FIG. 5 depicts a block diagram of an example computing system 500 according to example embodiments of the present disclosure. The example system 500 includes a computing system 502 and a machine learning computing system 532 that are communicatively coupled over a network 550.

In some implementations, the computing system 502 can perform the operations and functions of the various computing devices described herein. For example, the computing system 502 can represent wearable computing device, such as but not limited to a smart watch or an activity tracker, and/or other elements described herein and can perform the functions of such elements. The computing system 502 can include one or more distinct physical computing devices.

The computing system 502 can include one or more computing devices 504. The one or more computing devices 504 can include one or more processors 506 and a memory 508. The one or more processors 506 can be any suitable processing device (e.g., a processor core, a microprocessor, an ASIC, a FPGA, a controller, a microcontroller, etc.) and can be one processor or a plurality of processors that are operatively connected. The memory 508 can include one or more non-transitory computer-readable storage media, such as RAM, ROM, EEPROM, EPROM, one or more memory devices, flash memory devices, etc., and combinations thereof.

The memory 508 can store information that can be accessed by the one or more processors 506. For instance, the memory 508 (e.g., one or more non-transitory computer-readable storage mediums, memory devices) can store data 510 that can be obtained, received, accessed, written, manipulated, created, and/or stored. The data 510 can include, for instance, data indicative of: one or more calibration traces, one or more PPG traces, convolved signals, inputs/output, and/or any other data and/or information as described herein. In some implementations, the computing system 502 can obtain data from one or more memory device(s) that are remote from the system 502.

The memory 508 can also store computer-readable instructions 512 that can be executed by the one or more processors 506. The instructions 512 can be software written in any suitable programming language or can be implemented in hardware. Additionally, or alternatively, the instructions 512 can be executed in logically and/or virtually separate threads on processor(s) 506.

For example, the memory 508 can store instructions 512 that when executed by the one or more processors 506 cause the one or more processors 506 to perform any of the operations and/or functions described herein, including, for example, the operations and functions of any of the systems/apparatuses described herein, one or more portions of the methods/processes described herein, and/or any other functions or operations.

According to an aspect of the present disclosure, the computing system 502 can store or include one or more machine-learned models 516. As examples, the machine-learned models 516 can be or can otherwise include various machine-learned models such as, for example, neural networks (e.g., deep neural networks), support vector machines, decision trees, ensemble models, k-nearest neighbors models, Bayesian networks, or other types of models including linear models and/or non-linear models. Example neural networks include feed-forward neural networks, recurrent neural networks (e.g., long short-term memory recurrent neural networks), convolutional neural networks, or other forms of neural networks.

In some implementations, the computing system 502 can receive the one or more machine-learned models 516 from the machine learning computing system 532 over network 550 and can store the one or more machine-learned models 516 in the memory 508. The computing system 502 can then use or otherwise implement the one or more machine-learned models 516 (e.g., by processor(s) 506). In some implementations, the input to the machine-learned model(s) 516 can be a fused input based on signals from multiple sources.

The machine learning computing system 532 includes one or more processors 534 and a memory 536. The one or more processors 534 can be any suitable processing device (e.g., a processor core, a microprocessor, an ASIC, a FPGA, a controller, a microcontroller, etc.) and can be one processor or a plurality of processors that are operatively connected. The memory 536 can include one or more non-transitory computer-readable storage media, such as RAM, ROM, EEPROM, EPROM, one or more memory devices, flash memory devices, etc., and combinations thereof.

The memory 536 can store information that can be accessed by the one or more processors 534. For instance, the memory 536 (e.g., one or more non-transitory computer-readable storage mediums, memory devices) can store data 538 that can be obtained, received, accessed, written, manipulated, created, and/or stored. The data 538 can include, for instance, any of the data and/or information as described herein. In some implementations, the machine learning computing system 532 can obtain data from one or more memory device(s) that are remote from the machine learning computing system 532.

The memory 536 can also store computer-readable instructions 540 that can be executed by the one or more processors 534. The instructions 540 can be software written in any suitable programming language or can be implemented in hardware. Additionally, or alternatively, the instructions 540 can be executed in logically and/or virtually separate threads on processor(s) 534.

For example, the memory 536 can store instructions 540 that when executed by the one or more processors 534 cause the one or more processors 534 to perform any of the operations and/or functions described herein, including, for example, the operations and functions of any of the systems/apparatuses described herein, one or more portions of the methods/processes described herein, and/or any other functions or processes.

In some implementations, the machine learning computing system 532 includes one or more server computing devices. If the machine learning computing system 532 includes multiple server computing devices, such server computing devices can operate according to various computing architectures, including, for example, sequential computing architectures, parallel computing architectures, or some combination thereof.

Additionally, or alternatively, to the machine-learned model(s) 516 at the computing system 502, the machine learning computing system 532 can include one or more machine-learned models 542. As examples, the machine-learned models 542 can be or can otherwise include various machine-learned models such as, for example, neural networks (e.g., deep neural networks), support vector machines, decision trees, ensemble models, k-nearest neighbors models, Bayesian networks, or other types of models including linear models and/or non-linear models. Example neural networks include feed-forward neural networks, recurrent neural networks (e.g., long short-term memory recurrent neural networks), convolutional neural networks, or other forms of neural networks.

As an example, the machine learning computing system 532 can communicate with the computing system 502 according to a client-server relationship. For example, the machine learning computing system 532 can implement the machine-learned models 542 to provide a web service to the computing system 502. For example, the web service can provide the functions and operations of the optical sensing described herein.

Thus, machine-learned models 516 can be located and used at the computing system 502 and/or machine-learned models 542 can be located and used at the machine learning computing system 532.

In some implementations, the machine learning computing system 532 and/or the computing system 502 can train the machine-learned models 516 and/or 542 through use of a model trainer 544. The model trainer 544 can train the machine-learned models 516 and/or 542 using one or more training or learning algorithms. One example training technique is backwards propagation of errors. In some implementations, the model trainer 544 can perform supervised training techniques using a set of labeled training data. In other implementations, the model trainer 544 can perform unsupervised training techniques using a set of unlabeled training data. The model trainer 544 can perform a number of generalization techniques to improve the generalization capability of the models being trained. Generalization techniques include weight decays, dropouts, or other techniques.

In particular, the model trainer 544 can train a machine-learned model 516 and/or 544 based on a set of training data 546. The training data 546 can include, for example, labelled input data, etc. The model trainer 544 can be implemented in hardware, firmware, and/or software controlling one or more processors.

The computing system 502 can also include a communication interface 514 used to communicate with one or more systems or devices, including systems or devices that are remotely located from the computing system 502. The communication interface 514 can include any circuits, components, software, etc. for communicating with one or more networks 550. In some implementations, the communication interface 514 can include, for example, one or more of a communications controller, receiver, transceiver, transmitter, port, conductors, software and/or hardware for communicating data. Similarly, the machine learning computing system 532 can include a communication interface 548.

The network(s) 550 can be any type of network or combination of networks that allows for communication between devices. In some embodiments, the network(s) can include one or more of a local area network, wide area network, the Internet, secure network, cellular network, mesh network, peer-to-peer communication link and/or some combination thereof and can include any number of wired or wireless links. Communication over the network(s) can be accomplished, for instance, via a network interface using any type of protocol, protection scheme, encoding, format, packaging, etc.

FIG. 5 illustrates one example computing system 500 that can be used to implement the present disclosure. Other computing systems can be used as well. For example, in some implementations, the computing system 502 can include the model trainer 544 and the set of training data 546. In such implementations, the machine-learned models 516 can be both trained and used locally at the computing system 502. As another example, in some implementations, the computing system 502 is not connected to other computing systems.

In addition, components illustrated and/or discussed as being included in one of the computing systems 502 or 532 can instead be included in another of the computing systems 502 or 532. Such configurations can be implemented without deviating from the scope of the present disclosure. The use of computer-based systems allows for a great variety of possible configurations, combinations, and divisions of tasks and functionality between and among components. Computer-implemented operations can be performed on a single component or across multiple components. Computer-implemented tasks and/or operations can be performed sequentially or in parallel. Data and instructions can be stored in a single memory device or across multiple memory devices.

FIG. 6 illustrates an example of a photodetector 600. The photodetector 600 includes a first substrate 610 and a second substrate 630. The first substrate 610 includes a sensing area 612 (e.g., III-V material) that is electrically coupled to sensing circuitry 632 (e.g., CMOS circuitry) of the second substrate 630 via wire(s) 622 (e.g., wire-bonded).

FIG. 7 illustrates an example of a photodetector 700. The photodetector 700 includes a first substrate 710 and a second substrate 730, which can both be silicon substrates. The first substrate 710 and the second substrate 730 are wafer-bonded via a bonding interface 720 (e.g., oxide or any other suitable materials). The first substrate 710 includes multiple sensing areas 712a, 712b, . . . 712N, where N is a positive integer. In some implementations, the multiple sensing areas 712a, 712b, . . . 712N may be comprised of germanium that is deposited on the silicon substrate 710. The second substrate 730 includes multiple corresponding circuitry areas 732a, 732b, . . . 732N. The multiple sensing areas 712a, 712b, . . . 712N and the multiple corresponding circuitry areas 732a. 732b, . . . 732N are electrically coupled through the bonding interface 720 via wires 722.

Various means can be configured to perform the methods, operations, and processes described herein. For example, any of the systems and apparatuses (e.g., optical sensing apparatus and related circuitry) can include unit(s) and/or other means for performing their operations and functions described herein. In some implementations, one or more of the units may be implemented separately. In some implementations, one or more units may be a part of or included in one or more other units. These means can include processor(s), microprocessor(s), graphics processing unit(s), logic circuit(s), dedicated circuit(s), application-specific integrated circuit(s), programmable array logic, field-programmable gate array(s), controller(s), microcontroller(s), and/or other suitable hardware. The means can also, or alternately, include software control means implemented with a processor or logic circuitry, for example. The means can include or otherwise be able to access memory such as, for example, one or more non-transitory computer-readable storage media, such as random-access memory, read-only memory, electrically erasable programmable read-only memory, erasable programmable read-only memory, flash/other memory device(s), data register(s), database(s), and/or other suitable hardware.

While the disclosure has been described by way of example and in terms of a preferred embodiment, it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method for estimating a blood pressure of a user of a computing device, the method comprising:
obtaining, by one or more processors, at least one calibration trace, the at least one calibration trace being constructed to yield a fit between one or more selected reference traces and a corresponding parameter reference value for each of the one or more selected reference traces;
obtaining, by the one or more processors, a photoplethysmography (PPG) trace associated with a heartbeat cycle;
determining, by the one or more processors, a convolution of the PPG trace with the at least one calibration trace;
determining, by the one or more processors, a blood pressure estimate for the user based on the convolution of the PPG trace with the at least one calibration trace; and
providing, by the one or more processors, data representing the blood pressure estimate.

2. The method of claim 1, wherein:
the computing device is a wearable computing device comprising a sensor; and
the sensor obtains the PPG trace.

3. The method of claim 1, wherein the PPG trace is equal in size to the at least one calibration trace.

4. The method of claim 1, wherein the PPG trace includes a plurality of trace points, and wherein the plurality of trace points are sampled at arbitrarily chosen locations in the heartbeat cycle.

5. The method of claim 1, wherein the at least one calibration trace includes two calibration traces, the two calibration traces including: (i) a first calibration trace associated with systolic blood pressure values, and (ii) a second calibration trace associated with diastolic blood pressure values.

6. The method of claim 5, wherein the blood pressure estimate comprises an output from the convolution of the PPG trace and at least one of the first calibration trace and the second calibration trace.

7. The method of claim 1, wherein the PPG trace includes a plurality of trace points, the method further comprising:
determining, by the one or more processors, a weighted value for each trace point of the plurality of trace points based on the convolution;
determining, by the one or more processors, a region of interest in the PPG trace based on the determined weighted value for each trace point of the plurality of trace points; and
determining, by the one or more processors, a group of sampling points having a higher sampling rate in the region of interest for a future PPG trace.

8. The method of claim 1, wherein each calibration trace of the at least one calibration trace comprises a linear array of calibration coefficients representing weights of points associated with the one or more selected reference traces.

9. The method of claim 1, further comprising presenting the data to the user via a display of the computing device.

10. A computing device for estimating a blood pressure of a user, the computing device comprising:
one or more processors; and
a memory storing instructions for execution by the one or more processors to cause the one or more processors to perform operations, the operations comprising:
obtaining at least one calibration trace, the at least one calibration trace being constructed to yield a fit between one or more selected reference traces and a corresponding parameter reference value for each of the one or more selected reference traces;
obtaining a photoplethysmography (PPG) trace associated with a heartbeat cycle;
determining a convolution of the PPG trace with the at least one calibration trace;
determining a blood pressure estimate for the user based on the convolution of the PPG trace with the at least one calibration trace; and
providing data representing the blood pressure estimate.

11. The computing device of claim 10, wherein the computing device is a wearable computing device and further comprises a sensor, wherein the sensor obtains the PPG trace.

12. The computing device of claim 10, wherein the PPG trace includes a plurality of trace points, and wherein the plurality of trace points are sampled at arbitrarily chosen locations in the heartbeat cycle.

13. The computing device of claim 10, wherein the at least one calibration trace includes two calibration traces, the two calibration traces including: (i) a first calibration trace associated with systolic blood pressure values, and (ii) a second calibration trace associated with diastolic blood pressure values.

14. The computing device of claim 13, wherein the blood pressure estimate comprises an output from the convolution of the PPG trace and at least one of the first calibration trace and the second calibration trace.

15. The computing device of claim 10, wherein the PPG trace includes a plurality of trace points, the operations further comprising:
determining a weighted value for each trace point of the plurality of trace points based on the convolution;
determining a region of interest in the PPG trace based on the determined weighted value for each trace point of the plurality of trace points; and
determining a group of sampling points having a higher sampling rate in the region of interest for a future PPG trace.

16. A non-transitory, computer-readable medium storing instructions for execution by one or more processors to cause the one or more processors to perform operations, the operations comprising:
obtaining at least one calibration trace, the at least one calibration trace being constructed to yield a fit between one or more selected reference traces and a corresponding parameter reference value for each of the one or more selected reference traces;
obtaining a photoplethysmography (PPG) trace associated with a heartbeat cycle;
determining a convolution of the PPG trace with the at least one calibration trace;
determining a blood pressure estimate for a user based on the convolution of the PPG trace with the at least one calibration trace; and
providing data representing the blood pressure estimate.

17. The non-transitory, computer-readable medium of claim 16, wherein the data representing the blood pressure estimate is provided by a wearable computing device that further comprises a sensor, wherein the sensor obtains the PPG trace.

18. The non-transitory, computer-readable medium of claim 16, wherein the PPG trace is equal in size to the at least one calibration trace.

19. The non-transitory, computer-readable medium of claim 16, wherein the at least one calibration trace includes two calibration traces, the two calibration traces including a first calibration trace associated with systolic blood pressure values and a second calibration trace associated with diastolic blood pressure values.

20. The non-transitory, computer-readable medium of claim 16, wherein the PPG trace includes a plurality of trace points, the operations further comprising:
determining a weighted value for each trace point of the plurality of trace points based on the convolution;
determining a region of interest in the PPG trace based on the determined weighted value for each trace point of the plurality of trace points; and
determining a group of sampling points having a higher sampling rate in the region of interest for a future PPG.
